(19) United States
(12) Patent Application Publication
(10) Pub. No.: US 2018/0228437 A1
(43) Pub. Date: Aug. 16, 2018

(54) LOWER LIMB REHABILITATION SYSTEM

(71) Applicants: Kaohsiung Medical University, Kaohsiung (TW); National Chiao Tung University, Hsinchu City (TW)

(72) Inventors: Chia-Hsin Chen, Kaohsiung City (TW); Wei-Zen Chen, Hsinchu City (TW); Yi-Jen Chen, Kaohsiung City (TW); Li-Wei Ko, Hsinchu City (TW)

(21) Appl. No.: 15/895,524

(22) Filed: Feb. 13, 2018

Related U.S. Application Data
(60) Provisional application No. 62/459,249, filed on Feb. 15, 2017.

(51) Int. Cl. A61B 5/00; A63B 24/00; A61B 5/103; A61B 5/11

(52) U.S. Cl. CPC A61B 5/6807; A61B 5/4836; A63B 24/0006; A61B 2505/09; A61B 5/112; A61B 5/0022; A61B 2562/0247; A61B 5/1036

(57) ABSTRACT

A lower limb rehabilitation system includes an analysis platform, a smart insole, a motion sensor and a brain wave sensor. The analysis platform has a deep learning model. The smart insole generates plural pressure signals through plural pressure sensors of a pressure sensing film. The smart insole includes a processing unit controlling a transmission unit to transmit the pressure signals to the analysis platform. The processing unit is connected to a power supply unit. The motion sensor generates a motion signal. The brain wave sensor is coupled with the analysis platform and detects a brain wave signal. The analysis platform inputs the pressure signals, the motion signal and the brain wave signal into the deep learning model for analyzing a gait. The deep learning model analyzes whether the gait is correct. The analysis platform generates a warning message if the gait is analyzed to be incorrect.

### CROSS REFERENCE TO RELATED APPLICATION

The application claims the benefit of U.S. provisional application No. 62/459,249, filed on Feb. 15, 2017, and the entire contents of which are incorporated herein by reference.

### BACKGROUND OF THE INVENTION

### 1. Field of the Invention

The present invention generally relates to a lower limb rehabilitation system and, more particularly, to a lower limb rehabilitation system which can improve the accuracy in gait analysis of the lower limbs.

### 2. Description of the Related Art

Stroke has been among the top three leading causes of death. However, what is the most suffering to the patient is not the surgery process but the rehabilitation process. Gait analysis is the indicator method among all rehabilitation methods. As the continuous development of the medical technology, a variety of novel wearable devices have been developed to provide assistance in analyzing the gait of the patient. For example, the conventional device for assisting the gait analysis (such as a wearable device with an outer bracket) can include an angle sensor, a strain sensor and a pressure sensor that are embedded in a microprocessor, thereby assisting the patient in the rehabilitation process. However, due to the bulkiness of the outer bracket of the wearable device and the inconvenience in wearing and taking off the outer bracket, the use of the wearable device is inconvenient.

In light of this, it has been proposed by many researches that the sensors are embedded in an insole to detect whether the gait of the patient is correct and to make a treatment plan therefor. However, this mechanism simply analyzes the gait of the patient without using a physiological signal detector which can monitor the physiological signals of the patient during the movement of the limbs and the motor imagery while giving neurofeedbacks to the patient.

Thus, it is necessary to improve the conventional device.

### SUMMARY OF THE INVENTION

It is therefore the objective of this invention to provide a lower limb rehabilitation system which can detect the physiological signals of the patient for rehabilitation purposes of the lower limbs of the patient.

In an embodiment, a lower limb rehabilitation system includes an analysis platform, a smart insole, a motion sensor and a brain wave sensor. The analysis platform has a deep learning model. The smart insole generates a plurality of pressure signals through a plurality of pressure sensors of a pressure sensing film. The smart insole includes a processing unit controlling a transmission unit to transmit the plurality of pressure signals to the analysis platform. The processing unit is electrically connected to a power supply unit. The motion sensor is attached to the smart insole and is electrically connected to the processing unit. The motion sensor generates a motion signal transmitted to the analysis platform via the transmission unit. The brain wave sensor is coupled with the analysis platform and detects a brain wave signal. The analysis platform inputs the plurality of pressure signals, the motion signal and the brain wave signal into the deep learning model for analyzing a gait. The deep learning model analyzes whether the gait is correct. The analysis platform generates a warning message if the gait is analyzed to be incorrect.

Based on this, the lower limb rehabilitation system according to the invention can monitor the physiological signals of the patient during the movement of the limbs and the motor imagery, thereby improving the accuracy in gait analysis.

In an example, the processing unit and the power supply unit are disposed in an inner space of a heel portion of the smart insole. Thus, when the patient wears the shoe with the smart insole, more comfortable feeling can be provided.

In an example, the motion sensor includes at least one of an accelerometer, a gyroscope and an electronic compass. Thus, the accuracy in gait analysis can be further improved.

In an example, the lower limb rehabilitation system further includes an electromyography detector coupled with the analysis platform and detecting a lower limb muscle signal. The analysis platform inputs the lower limb muscle signal into the deep learning model for analyzing the gait. Thus, the accuracy in gait analysis can be further improved.

In an example, the lower limb rehabilitation system further includes a plurality of functional electrical stimulus units disposed on a first face of the smart insole and electrically connected to the processing unit and the power supply unit. If the gait is analyzed to be incorrect, the analysis platform obtains a functional electrical stimulation signal from the deep learning model and uses the processing unit to control the plurality of functional electrical stimulus units to generate a functional electrical stimulation according to the functional electrical stimulation signal. Thus, the rehabilitation effect can be improved.

In an example, after the functional electrical stimulation signal is generated, the analysis platform controls the brain wave sensor to obtain another brain wave signal. The analysis platform determines whether an energy of a a wave of the other brain wave signal is smaller than an energy of a a wave of the brain wave signal. The analysis platform generates the warning message if the determined result is negative. Thus, the rehabilitation effect can be further improved.

### BRIEF DESCRIPTION OF THE DRAWINGS

The present invention will become more fully understood from the detailed description given hereinafter and the accompanying drawings which are given by way of illustration only, and thus are not limitative of the present invention, and wherein:

FIG. 1 shows a block diagram of a lower limb rehabilitation system of an embodiment according to the invention.

FIG. 2 shows an insole of the lower limb rehabilitation system of the embodiment according to the invention.

In the various figures of the drawings, the same numerals designate the same or similar parts. Furthermore, when the terms “first”, “second”, “inner”, “outer”, “length” and similar terms are used hereinafter, it should be understood that these terms have reference only to the structure shown in the drawings as it would appear to a person viewing the drawings, and are utilized only to facilitate describing the invention.

### DETAILED DESCRIPTION OF THE INVENTION

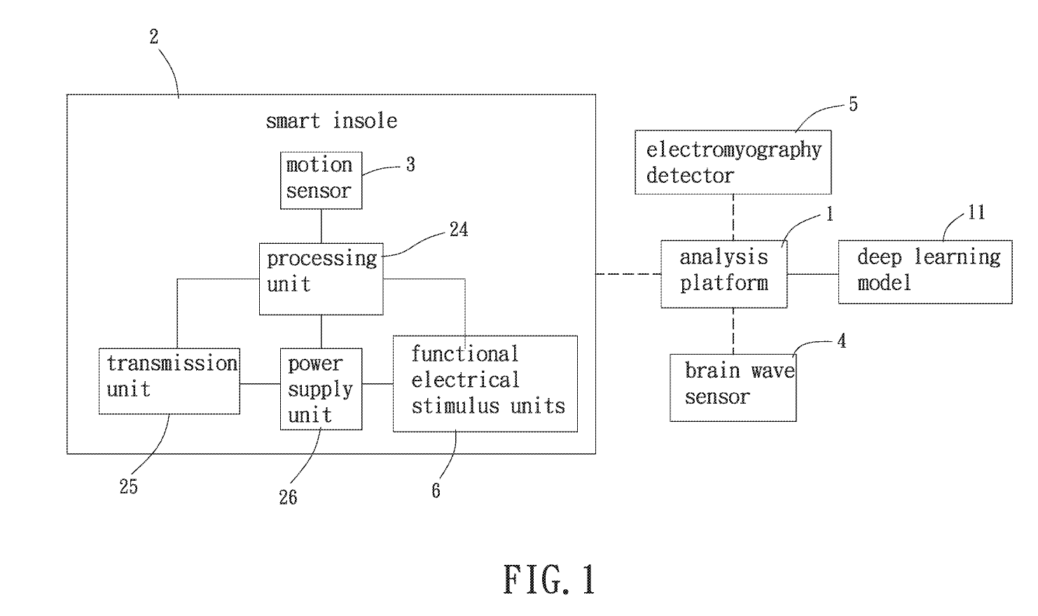

FIG. 1 shows a lower limb rehabilitation system of an embodiment according to the invention. The lower limb rehabilitation system includes an analysis platform 1, a smart insole 2, a motion sensor 3 attached to the smart insole 2, and a brain wave sensor 4 coupled with the analysis platform 1.

The analysis platform 1 can be any device having a data processing function, a signal generation function and a control function, such as a micro control unit (MCU). The analysis platform 1 includes a deep learning model 11 configured to analyze the gait of a patient who is walking. The analysis platform 1 is trained by a convolutional neural network with deep learning, as it can be readily appreciated by the skilled persons.

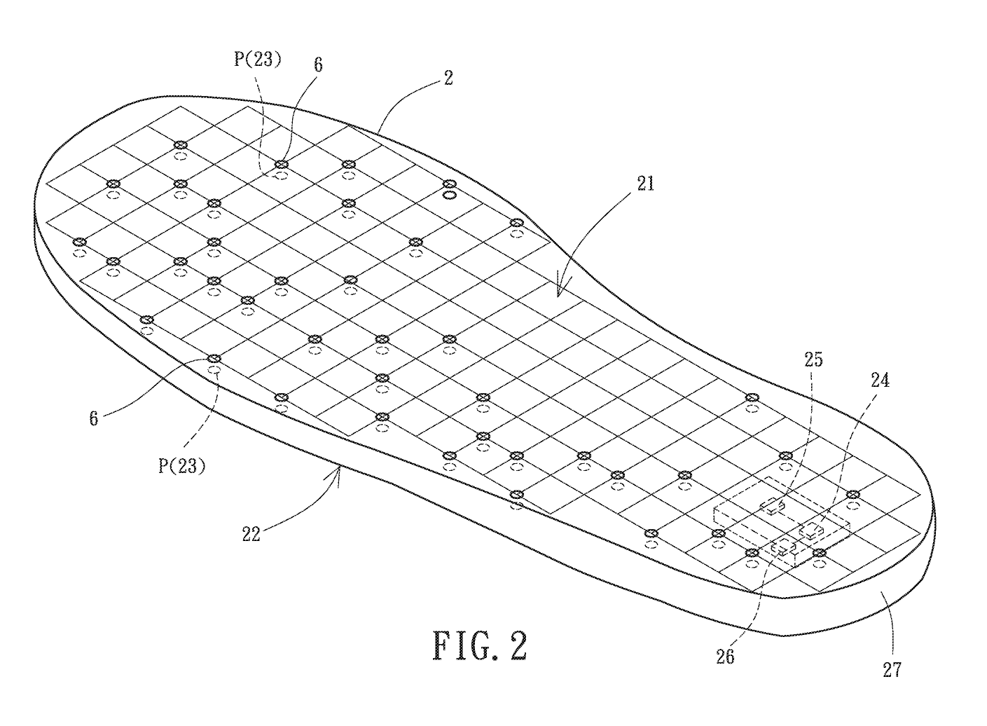

Referring to FIG. 2, the style and the manufacturing method of the smart insole 2 are not limited in this invention. For example, the smart insole 2 can be printed by a 3D printer that scans the sole shape of the foot of the patient. In a preferred case, the smart insole 2 is made of a flexible material. Specifically, the smart insole 2 includes a first face 21 and a second face 22. The smart insole 2 further includes a pressure sensing film 23 and a plurality of pressure sensors P located on the pressure sensing film 23. When the patient is walking, the smart insole 2 detects the pressure applied to the pressure sensors P, thereby generating a plurality of pressure signals. In a preferred case, the pressure sensing film 23 is disposed on the second face 22. The smart insole 2 controls a transmission unit 25 to send the pressure signals to the analysis platform 1 through a processing unit 24.

In this embodiment, the transmission unit 25 may be a wireless transmission module such as WiFi, ZigBee, Bluetooth or infrared, but is not limited thereto. Besides, the processing unit 24 is electrically connected to a power supply unit 26 that supplies power to the processing unit 24. In a preferred case, the power supply unit 26 may be a piezoelectric film such that the electricity can be generated by the pressure of the foot stepping on the ground when the patient is walking. The processing unit 24, the transmission unit 25 and the power supply unit 26 can be preferably disposed in an inner space of a heel portion 27 of the smart insole 2.

The motion sensor 3 is electrically connected to the processing unit 24 of the smart insole 2 and is configured to sense a motion signal generated by the walk of the patient. The motion signal is transmitted to the analysis platform 1 through the transmission unit 25. The motion sensor 3 can include at least one of an accelerometer, a gyroscope and an electronic compass.

The brain wave sensor 4 (EEG) is coupled with the analysis platform 1 and detects a brain wave signal during the walk of the patient. In this embodiment, the brain wave signal is from a motor area of the brain. The analysis platform 1 inputs the pressure signals, the motion signal and the brain wave signal to the deep learning model 11 for gait analysis. The deep learning model 11 analyzes whether the gait of the patient is correct. In this embodiment, the deep learning model 11 can analyze a heel-strike time, a toe-off time, a stance phase, a stride length and a step length of the patient according to the pressure signals, and analyze the change in a pitch of the patient according to the motion signal. This can be readily appreciated by the skilled persons and therefore are not described again. If it is determined that the gait of the patient is incorrect, the analysis platform 1 generates a warning message to remind the patient of incorrect gait. The message can be sent to the handset of the patient, but is not limited thereto.

In addition, the lower limb rehabilitation system according to the invention may further include an electromyography (EMG) detector 5 coupled with the analysis platform 1. The electromyography detector 5 is configured to detect a lower limb muscle signal of the patient when the patient is walking. The analysis platform 1 inputs the pressure signals, the motion signal, the brain wave signal and the lower limb muscle signal to the deep learning model 11 for gait analysis.

The lower limb rehabilitation system according to the invention may further include a plurality of functional electrical stimulus units 6. The functional electrical stimulus units 6 are mounted on the first face 21 of the smart insole 2 in order to make contact with the sole of the patient. Preferably, the functional electrical stimulus units 6 are disposed in the locations respectively corresponding to the acupuncture points of the sole. The functional electrical stimulus units 6 are electrically connected to the processing unit 24 and the power supply unit 26. The analysis platform 1 analyzes whether the gait of the patient is correct. If not, the analysis platform 1 obtains a functional electrical stimulation signal from the deep learning model 11, and uses the processing unit 24 to control the functional electrical stimulus units 6 to generate a functional electrical stimulation according to the functional electrical stimulation signal. Thus, the nerve of the acupuncture point can be stimulated.

Based on the above, after the nerves of the acupuncture points of the sole are stimulated by the functional electrical stimulus units 6, the analysis platform 1 may determine the rehabilitation effect of the functional electrical stimulation based on the activation level of the motor cortex or the sensorimotor cortex. As an example of analyzing the activation level of the motor cortex of the brain, the analysis platform 1 obtains a secondary brain wave signal of the patient through the brain wave sensor 4. The analysis platform 1 determines whether the energy of the a wave of the secondary brain wave signal is smaller than the energy of the a wave of the brain wave signal. In a preferred case, the energy difference between the a waves of the secondary brain wave signal and the brain wave signal is smaller than 3 dB. If so, it shows that the functional electrical stimulation has an excellent rehabilitation effect. If not, the analysis platform 1 generates the warning message to notify the patient of incorrect gait.

In conclusion, the lower limb rehabilitation system according to the invention can monitor the physiological signals of the patient during the movement of the limbs and the motor imagery, thereby improving the accuracy in gait analysis. Besides, through the functional electrical stimulus units that apply functional electrical stimulation to the nerves of the acupuncture points of the sole, the lower limb rehabilitation system according to the invention can have an improved rehabilitation effect.

Although the invention has been described in detail with reference to its presently preferable embodiments, it will be understood by one of ordinary skill in the art that various modifications can be made without departing from the spirit and the scope of the invention, as set forth in the appended claims.

What is claimed is:

1. A lower limb rehabilitation system comprising:
an analysis platform having a deep learning model;
a smart insole generating a plurality of pressure signals through a plurality of pressure sensors of a pressure sensing film, wherein the smart insole includes a processing unit controlling a transmission unit to transmit the plurality of pressure signals to the analysis platform, and wherein the processing unit is electrically connected to a power supply unit;
a motion sensor attached to the smart insole and electrically connected to the processing unit, wherein the motion sensor generates a motion signal transmitted to the analysis platform via the transmission unit; and
a brain wave sensor coupled with the analysis platform and detecting a brain wave signal, wherein the analysis platform inputs the plurality of pressure signals, the motion signal and the brain wave signal into the deep learning model for analyzing a gait, wherein the deep learning model analyzes whether the gait is correct, and wherein the analysis platform generates a warning message if the gait is analyzed to be incorrect.

2. The lower limb rehabilitation system as claimed in claim 1, wherein the plurality of pressure sensors is disposed on the smart insole in locations corresponding to acupuncture points of a sole of a user, respectively.

3. The lower limb rehabilitation system as claimed in claim 1, wherein the processing unit and the power supply unit are disposed in an inner space of a heel portion of the smart insole.

4. The lower limb rehabilitation system as claimed in claim 1, wherein the motion sensor includes at least one of an accelerometer, a gyroscope and an electronic compass.

5. The lower limb rehabilitation system as claimed in claim 1, further comprising an electromyography detector coupled with the analysis platform and detecting a lower limb muscle signal, wherein the analysis platform inputs the lower limb muscle signal into the deep learning model for analyzing the gait.

6. The lower limb rehabilitation system as claimed in claim 1, further comprising a plurality of functional electrical stimulus units disposed on a first face of the smart insole and electrically connected to the processing unit and the power supply unit, wherein, if the gait is analyzed to be incorrect, the analysis platform obtains a functional electrical stimulation signal from the deep learning model and uses the processing unit to control the plurality of functional electrical stimulus units to generate a functional electrical stimulation according to the functional electrical stimulation signal.

7. The lower limb rehabilitation system as claimed in claim 6, wherein the analysis platform controls the brain wave sensor to obtain another brain wave signal, wherein the analysis platform determines whether an energy of a α wave of the other brain wave signal is smaller than an energy of a α wave of the brain wave signal, and wherein the analysis platform generates the warning message if the determined result is negative.
